(19) United States
(12) Patent Application Publication
(10) Pub. No.: US 2008/0160111 A1
(43) Pub. Date: Jul. 3, 2008

(54) Type I Allergy Inhibitor and Methods of Inhibiting the Onset of Type I Allergy Using Fulvic Acid

(76) Inventors: Hiroko Isoda, Ibaraki (JP); Parida Yamada, Ibaraki (JP); Yukuo Abe, Ibaraki (JP)

Correspondence Address: JORDAN AND HAMBURG LLP, 122 EAST 42ND STREET, SUITE 4000, NEW YORK, NY, 10168, US

(21) Appl. No.: 11/885,217

(22) PCT Filed: Jan. 5, 2006
(86) PCT No.: PCT/JP06/00026

§ 371 Date: Oct. 25, 2007

(30) Foreign Application Priority Data
Feb. 28, 2005 (JP) 2005-053656

(51) Int. Cl. A61K 36/00; A61P 37/08

(52) U.S. Cl. 424/725

(57) ABSTRACT

The present invention aims to elucidate the relationship between fulvic acid contained in a humic substance and the onset mechanism of the type I allergy, and inhibits the onset of the type I allergy using the fulvic acid.

The present invention provides a type I allergy inhibitor which inhibits an antigen sensitization phase or/and an antibody sensitization phase, and the degranulation phase of cells with fulvic acid and a composition containing at least the fulvic acid. Moreover, the present invention provides a method for inhibiting the onset of the type I allergy by performing a specific hyposensitization treatment using the fulvic acid and, in particularly, a method for inhibiting an antigen sensitization phase or/and an antibody sensitization phase with fulvic acid. Or, the present invention provides a method for inhibiting the onset of the type I allergy by performing a nonspecific hyposensitization treatment using fulvic acid.

### BACKGROUND OF THE INVENTION

The present invention relates to a technique for inhibiting the onset of the type I allergy.

The “humic substance” is an organic component generated by plant decay. According to the definition in the International Humic Substance society (abbreviated as IHSS), the humic substance refers to a fraction obtained by extracting the soil with alkali, such as sodium hydroxide, or a fraction which adsorbs to an XAD resin with natural water and is eluted with an aqueous dilute alkaline solution. Among the fractions, a fraction which is precipitated by acid is referred to as a “humic acid (humic acid)” and a fraction which is not precipitated is referred to as “fulvic acid”, respectively.

The fulvic acid relating to the theme of the present invention is known to serve as a source of supplying iron to the sea due to the characteristic that the fulvic acid strongly bonds to iron or is known to have environment cleanup functions, such as coagulation ability (complex-forming ability) of harmful heavy metals, such as copper or the like, reduction detoxification of hexavalent chromium which is a carcinogen, dechlorination of an organic halide, etc. However, the details of various functions which are presumably given to the fulvic acid have not yet been elucidated.

In recent years, studies on physiological actions of fulvic acid exerted on the living body in addition to the above-mentioned environmental cleanup functions are gradually carried out. For example, Patent Document 1 discloses pharmaceutical compositions comprising fulvic acid (especially oxyfulvic acid), salts thereof, esters thereof, or derivatives thereof, and particularly pharmaceutical compositions effective for diseases, such as inflammation; acne; eczema; infections of bacteria, fungus, or virus; etc. Examples shown in Patent Document 1 describe the action of oxyfulvic acid exerted on the production of oxidant due to human neutrophile leukocyte, evaluation of the anti-inflammatory property of oxyfulvic acid by applying pharmaceutical cream comprising oxyfulvic acid to inflammation parts of mice, evaluation of the efficacy of pharmaceutical compositions comprising oxyfulvic acid to purulent traumatic dermatitis of cats and dogs, oral toxicity confirmatory test of pharmaceutical compositions comprising oxyfulvic acid using laboratory animals, anti-microorganism confirmatory test using a pharmaceutical cream agent comprising oxyfulvic acid, etc., and mainly confirms the anti-inflammatory effect of oxyfulvic acid when applied to a dermatological agent.

Patent Document 2 discloses the antibacterial action; antiallergic action; antiinflammatory action; hypoallergenic property and itching alleviating property for the allergy skin and atopy skin; etc., of multi-ingredient containing cosmetic materials comprising a fulvic acid eluted from humus, which is a water-soluble humic substance, enzyme, vitamins, amino acids, minerals, etc., and also discloses the application thereof to a cosmetic raw material for use in the mouth. However, Examples shown in Patent Document 2 merely disclose confirmatory tests for the stimulus to the skin, antimicrobial activity, oral administration toxicity, active oxygen elimination activity, etc.

Patent Document 3 discloses an antibacterial water comprising, as an active ingredient, an extract which is extracted from humus with water, dilute alcohol, etc., and whose pH value is 4.0 or less. Patent Document 3 discloses the growth inhibitory effect against oral bacteria, in which the above-mentioned extract causes a bad tooth and periodontosis; food poisoning causative bacillus, such as Staphylococcus aureus or the like; etc.
Patent Document 1: Translation of Japanese Patent Publication No. 2002-526407
Patent Document 2: Japanese Unexamined Patent Publication No. 2003-267821
Patent Document 3: Japanese Unexamined Patent Publication No. H06-87752

### SUMMARY OF THE INVENTION

Patent Documents 1 to 3 above merely disclose subjectively the antibacterial action and/or antiinflammatory action of fulvic acid, and nowhere disclose nor suggest a direct causal relationship between the fulvic acid and the type I allergy, and, more particularly, actions of the fulvic acid relating to hyposensitization in the onset mechanism of the type I allergy.

In view of the above, a primary object of the present invention is to provide a technique of inhibiting the onset of the type I allergy based on the hyposensitization of the fulvic acid by elucidating the relationship between the fulvic acid contained in a humic substance and the onset mechanism of the type I allergy.

First, the “type I allergy” which is the theme of the present invention is one type of allergic reactions, and is also referred to as anaphylaxis allergy or immediate allergy. In this type I allergy, when antibodies, such as an IgE antibody or the like produced when exposed to an antigen, are strongly bonded to receptors on the surfaces of mast cells or basophilic leucocytes at the Fc site, the receptors are crosslinked to transform the cell surfaces; various enzymes are activated and calcium ions (Ca²⁺) flow into cells to emit granulations out of the cells (degranulation); and then, mediators (chemical transmitters), such as enzymes (e.g., histamine, serotonine, β-hexosaminidase, etc.) are emitted from the granulations. These mediators accelerate the permeability of a blood capillary and cause inflammation on the tunica mucosa nasi, tunica mucosa bronchiorum, skin, etc. The symptoms of allergic rhinitis, bronchial asthma, and atopic dermatitis are caused by this type I allergy.

The present invention elucidates the relationship between the developmental mechanism of the type I allergy and the fulvic acid, and provides a technique of inhibiting the onset of the type I allergy based on the specific hyposensitization of fulvic acid.

Specifically, the present invention provides a type I allergy inhibitor which inhibits an antigen sensitization phase or/and an antibody sensitization phase with the fulvic acid, or a type I allergy inhibitor which inhibits the degranulation phase of cells with the fulvic acid. In particular, the present invention provides an type I allergy inhibitor which inhibits the degranulation phase by avoiding calcium ions (Ca²⁺) from flowing into the cells. Any substances extracted from, for example, moss peat can be suitably used for the fulvic acid for use in such inhibitors.

Moreover, the present invention provides a composition comprising at least fulvic acid which inhibits the type I allergy in the antigen sensitization phase or/and the antibody sensitization phase. This composition can demonstrate its effects when mixed in, for example, cosmetics, such as skin care cosmetics (e.g., facial wash, facial cream, facial pack, etc.), dermatological agent, poultice, other medical drugs, quasi medical drugs, foods and drinks, etc. As an ingredient of such products, ingredients other than a fulvic acid-containing composition are freely mixed according to objects and applications of the products. For example, mentioned are facial wash in which fulvic acid is combined with an ingredient adsorbing pore-clogging debris, facial cream and facial pack in which a moisturizing ingredient, such as collagen or the like, is combined with fulvic acid, medical drugs in which other medicinal ingredients are combined with fulvic acid, foods and drinks in which another functional ingredients are combined with fulvic acid, etc.

Furthermore, the present invention provides a method for inhibiting the onset of the type I allergy by performing a specific hyposensitization treatment using fulvic acid, and, more particularly, provides a method for inhibiting the onset of the type I allergy by suppressing the antigen sensitization phase, or/and antibody sensitization phase with fulvic acid. Or, the present invention provides a method for inhibiting the onset of the type I allergy by performing a nonspecific hyposensitization treatment using fulvic acid, and, in particular, provides a method for inhibiting the onset of the type I allergy by suppressing degranulation using fulvic acid. The effects of such methods can be acquired by administering fulvic acid in the form of drugs, foods and drinks, etc., in the living body or by contacting fulvic acid to the skin by, for example, applying, pasting, spraying, or the like.

Hereinafter, the main technical terms relevant to the present invention are described.

First, the “fulvic acid” refers to fulvic acid contained in a fraction obtained from a humic substance as a raw material according to the procedure (described in Examples later) prescribed by the International Humic Substances Society (IHSS).

The “moss peat (peat)” is a suitable raw material for extracting the fulvic acid. The moss peat is generated when various plants grow, and wither and die in a swamp or a damp area, and are deposited in an imperfect condition underwater, where oxygen is not sufficiently supplied, at relatively low temperatures for several hundreds to tens of thousands. Such moss peat is generated when plant residues are placed for a long period of time under the conditions where plant residues are hard to be oxidized because they are completely soaked in water and do not contact air, and where plant residues are very hard to be decomposed because they are not affected by the action of aerobic bacteria and are relatively slightly affected, if any, by the action of anaerobic bacteria. This process is referred to as humification, and the essential nature of the process is not thoroughly clarified, but it is thought from the viewpoint of chemical reaction that hydrolysis is a main process and decarboxylation also occurs. The organic ingredient of moss peat is essentially formed of five kinds of elements of carbon, hydrogen, oxygen, nitrogen, and sulfur. The elementary composition of moss peat reflects the decomposition characteristics of the organic substance in the formation process of moss peat. In general, when decomposition of moss peat progresses, the contents of carbon, hydrogen, sulfur, and nitrogen, which are ingredients of the moss peat organic component, increase, and the content of oxygen decreases. Studies on the elementary composition of the moss peat organic component are thoroughly carried out, and it is confirmed that the elementary composition is midway between timber and lignite. The elementary composition of the moss peat organic component in which the decomposition is weak is close to timber, and the elementary composition of the moss peat organic component in which decomposition progressed is close to lignite.

The “antigen sensitization phase” means the phase of the antigen-antibody reaction in the course of the above-described developmental mechanism of the type I allergy.

The “antibody sensitization phase” means the phase in which an IgE antibody is bonded to a receptor in the course of the above-described developmental mechanism of the type I allergy.

The “specific hyposensitization” refers to inhibiting or intercepting any stage in the process in the course of applying a stimulus to mast cells or basophilic leucocytes from antibodies, such as IgE antibodies, produced by antigens.

The “nonspecific hyposensitization” refers to inhibiting or suppressing the degranulation of mast cells or basophilic leucocytes, i.e., inhibiting or suppressing release of mediators (chemical transmitters) from mast cells or basophilic leucocytes.

The present invention can inhibit the onset of the type I allergy. In particular, the antigen sensitization phase or/and antibody sensitization phase of the type I allergy reaction can be inhibited by fulvic acid, or the degranulation phase of cells can be inhibited by fulvic acid.

When a composition containing the fulvic acid is used for products which act on the skin, such as facial wash, cosmetics, etc., various effects, such as whitening effect, moisturizing effect, moisture penetration enhancement effect of a cosmetic lotion, penetration enhancement effect of a cosmetic ingredient, effect of preventing drying, etc., can also be acquired in addition to the inhibitory effect of the onset of the type I allergy.

### BRIEF DESCRIPTION OF THE DRAWINGS

FIG. 1 is a flow chart illustrating a method for extracting fulvic acid which is prescribed by the International Humic Substances Society (IHSS).

FIG. 2 is a table substituting for a drawing showing the analysis results of the elementary composition of fulvic acid.

FIG. 3 is a view (graph) showing the FT-IR spectra of fulvic acids (FA) whose raw materials are different from each other.

FIG. 4 is a view (graph) showing the hydrogen nuclear magnetic resonance (¹H-NMR) spectra of fulvic acids (FA) whose raw materials are different from each other.

FIG. 5 is a view showing the ¹³C-CP/MASNMR spectra of humic acids (HA) and fulvic acids (FA) extracted from each of Canadian moss peat and weathered coal.

FIG. 6 is a table substituting a drawing which summarizes the physical properties analysis results of each fulvic acid extracted from each raw material of original underground brine, waste underground brine, weathered coal, Canadian moss peat, and andosol.

FIG. 7 is a table substituting for a drawing showing the characteristics of the fulvic acid originating from Canadian moss peat (CP-FA) used in the bioassay of Example 3.

FIG. 8 is view (graph) showing ¹³C-CP/MASNMR spectrum of humic acid (HA) and fulvic acid (FA) of Canadian moss peat.

FIG. 9 is a view (graph) showing the results of investigating the influence of fulvic acid (CP-FA) on β-hexosaminidase release inhibition rate.

FIG. 10 is a view (graph) showing the results of measuring the binding inhibition of a receptor and an antibody.

FIG. 11 is a view (graph group) showing the results of investigating the influence of fulvic acid on Ca²⁺ inflow inhibition due to antigen stimulus.

FIG. 12 is a view for schematically illustrating the relationship of the developmental mechanism of the type I allergy and fulvic acid.

### DETAILED DESCRIPTION OF THE INVENTION

### EXAMPLE 1

### <Method for Extracting Fulvic Acid>

In the experiment, fulvic acid of an XAD-7 resin adsorption fraction was extracted from moss peat according to the procedure prescribed by the International Humic Substance Society (IHSS), which is shown in FIG. 1. Canadian moss peat (Sphagnum(moss) peat, product of Fisons Horticulture, Inc.) was used as moss peat.

The Sphagnum (moss) peat as a raw material was subjected to an acid treatment with 0.1 mol L⁻¹ of hydrochloric acid (HCL). The insoluble component was extracted with 1 mol L⁻¹ of an aqueous sodium hydroxide (NaOH) solution, and 6 mol L⁻¹ of hydrochloric acid was added to a supernatant liquid obtained by centrifugal separation to precipitate humic acid (HA).

Aqueous solutions obtained in all the processes were adsorbed to an XAD-7 resin (ORGANO Co.), and desalting was carried out on an AGMP-50 cation exchange resin (Bio-RadLaboratories). Then, a freeze-dried resultant was used as fulvic acid. The extraction ratio of the fulvic acid from the Sphagnum(moss) peat as a raw material was 6.4% by calculating from a weight ratio of a dried fulvic acid after extraction to a dried raw material.

### EXAMPLE 2

### <Analysis of Fulvic Acid>

In order to understand the properties of the fulvic acid relating to the present invention, chemical analyses of fulvic acid obtained from each of waste underground brine, weathered coal, Sphagnum (moss) peat, and andosol were conducted.

(1) Analysis of Elementary Composition

Carbon (C), hydrogen (H), and nitrogen (N) of fulvic acid were measured using a CHN analyzer (YANACO CHN CORDERMT-5), and calculated as (composition) weight percent per (all) weight in which the total ash was subtracted, and oxygen was calculated as the remainder. The elementary composition value was expressed as dry ash free basis (d.a.f).

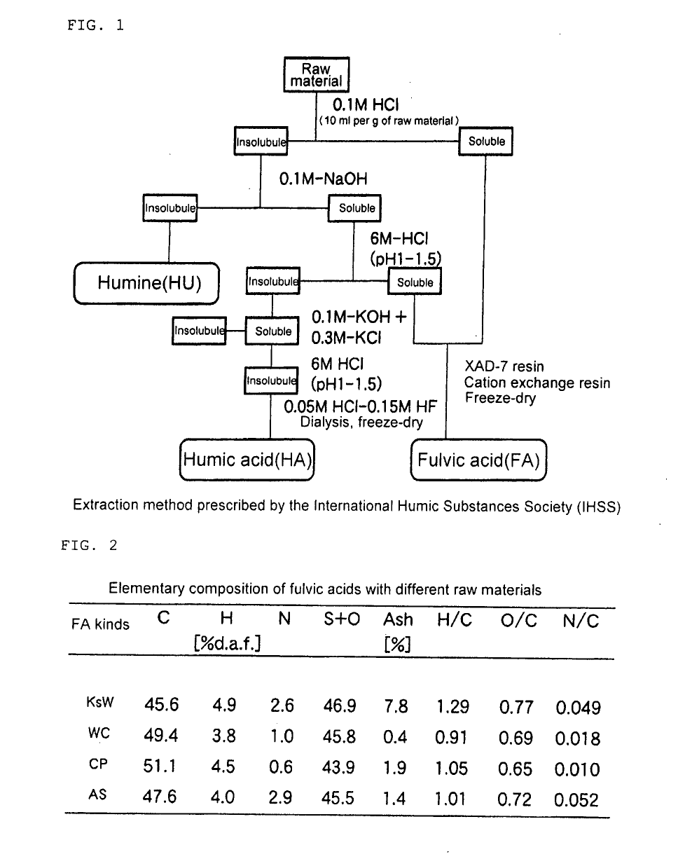

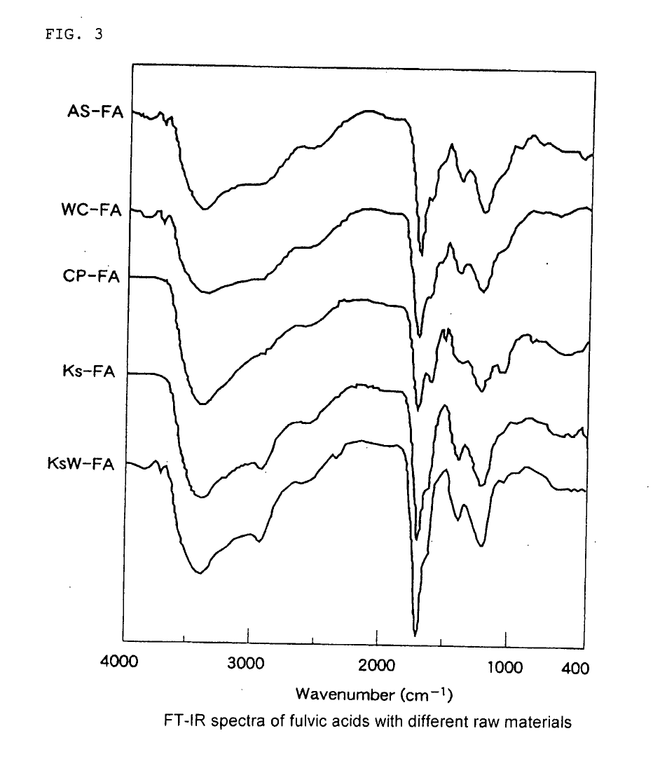

The above-mentioned analysis results about the elementary composition are shown in FIG. 2. In FIG. 2, “KsW” represents waste underground brine, “WC” represents weathered coal, “CP” represents Canadian moss peat, and “AS” represents andosol, respectively.

(2) Fourier Transform Infrared (FT-IR) Analysis

Measurement of the Fourier transform infrared of fulvic acid was carried out using a JASCOFT/IR-3 spectrometer based on a known KBr tablet method. A tablet was formed using 1 mg of sample and 100 mg of KBr, and the absorption spectrum of 400 to 4000 cm⁻¹ was measured.

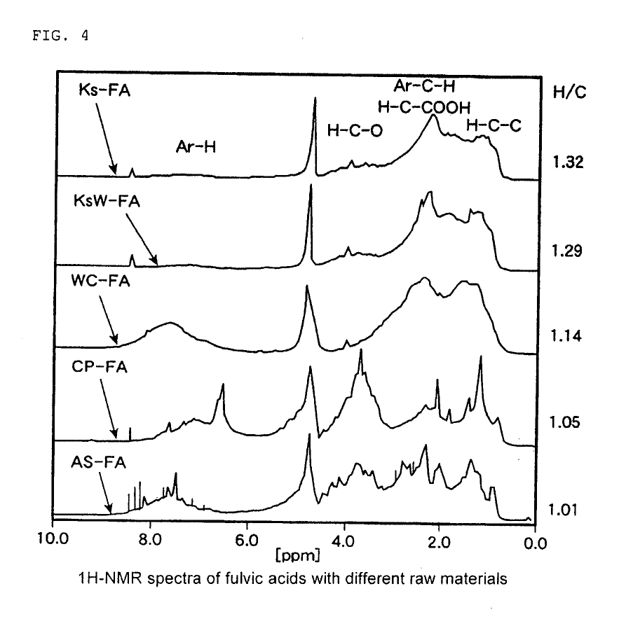

FIG. 3 shows the Fourier transform infrared (FT-IR) of fulvic acids (FA) extracted from various different raw materials. In FIG. 3, “Ks” represents an original underground brine, “KsW” represents waste underground brine, “WC” represents weathered coal, “CP” represents Canadian moss peat, and “AS” represents andosol, respectively.

As is clear from FIG. 3, with respect to the absorption near 3400 cm⁻¹, almost all fulvic acids showed strong absorption due to the hydrogen-bonded OH groups, but the absorption of the weathered coal FA (WC-FA) was relatively weak. In the absorption near 2900 cm⁻¹, which presumably originates from aliphatic carbons, no notable differences were observed in each fulvic acid of soil (AS), moss peat (CP), and weathered coal (WC), and in contrast, a clear absorption was observed in the underground brine FA (Ks-FA, KsW-FA). Two absorptions were observed near 1720 cm⁻¹ and 1620 cm⁻². The former mainly represents the absorption due to C=0 stretching of a carboxyl group, and presumably slightly shows absorption due to a carbonyl. To the latter, two structures of the absorptions originating from a hydrogen-bonded carbonyl group and a conjugated C═C bond with an aromatic structure presumably contribute. With respect to the absorption near 1720 cm⁻¹, the absorption of the weathered coal FA (WC-FA) is slightly stronger than that of the moss peat FA (CP-FA), and the absorption of underground brine FA was remarkable. With respect to the absorption near 1620 cm⁻¹, almost all the samples merely showed relatively weak absorption, and this is in agreement with the fact that the amount of quinone groups is smaller in fulvic acid than in humic acid. The absorption spectrum near 1400 cm⁻¹ and a relatively broad spectrum of a 1300 to 1200 cm⁻¹ region presumably originate from the C—O stretching vibration of a carboxyl group and deformation vibration of OH. Although absorption is observed in all the fulvic acids, the absorption intensity of underground brine FA is relatively strong compared with that of moss peat, soil, and weathered coal FA. A small trough near 800 cm⁻¹ is clearly observed in almost all the samples. This is presumably C—H deformation vibration on an aromatic ring. Thus, based on the results of Fourier transform infrared, it is concluded that fulvic acids (FA) extracted from various different raw materials are coagulation of a series of compounds having the same fundamental chemical structure.

FIG. 4 shows the hydrogen nuclear magnetic resonance (¹H-NMR) spectrum of fulvic acids (FA) obtained from various different raw materials. With respect to the parameter of the spectrum, the 0 to 1.9 ppm region represents aliphatic proton, 1.9 to 3.2 ppm region represents hydrogen which is mainly connected to a carboxyl carbon, 3.2 to 6.2 ppm region represents attributive hydrogen, such as hydrocarbon carbon, and 6.2 to 8.6 ppm region represents aromatic carbon attributive hydrogen, respectively.

It was clarified from the measurement results that, in the fulvic acid (Ks-FA) originating from original underground brine and/or the fulvic acid (KsW-FA) originating from extracted underground brine, the peak of aliphatic hydrogen attribution appearing near 0 to 1.9 ppm region is remarkable, which showed that a large amount of aliphatic group is contained. In contrast, it was clarified that, in the fulvic acid (WC-FA) originating from weathered coal, the peak near 6.2 to 8.6 ppm region showing aromatic hydrogen attribution is strong, which showed that a large amount of aromatic group is contained (see FIG. 4).

Moreover, relatively many composite spectra were obtained in the fulvic acid (CP-FA) originating from Canadian moss peat similarly as in the fulvic acid (AS-FA) originating from andosol. Thus, although the fulvic acid originating from Canadian moss peat (CP-FA) is inferior to the fulvic (KsW-FA) originating from extracted underground brine, the fulvic acid originating from Canadian moss peat (CP-FA) comprises a large amount of aliphatic hydrogen and carbohydrate hydrogen compared with the fulvic originating from weathered coal (WC-FA). Thus, it was found that the fulvic acid originating from Canadian moss peat (CP-FA) was a fulvic acid at a relatively low decomposition stage (see FIG. 4).

(3) ¹³C Nuclear Nuclear Magnetic Resonance Spectrum (¹³C-NMR) Analysis

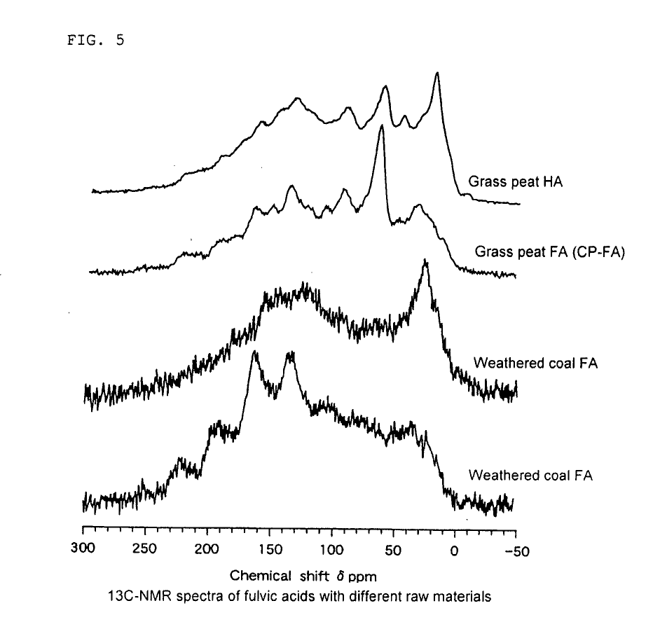

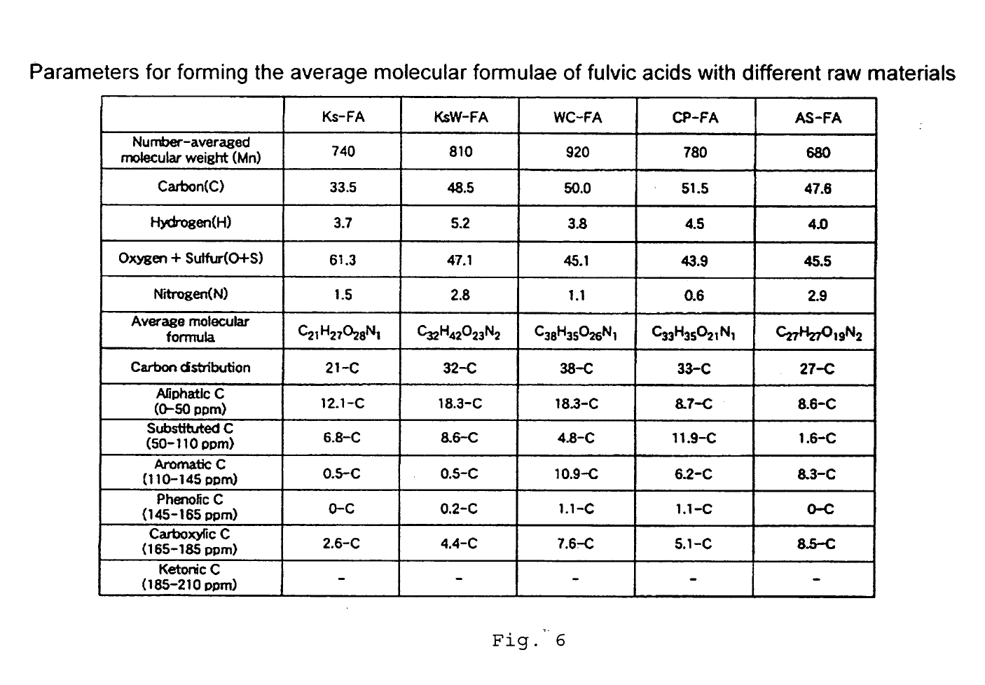

With respect to ¹³C nuclear nuclear magnetic resonance spectrum, fine particle measurement was performed using BRUKERAMX-400 spectrometer by a known cross polarization and magic angle spinning (CP/MAS) method. The measurement frequency was 100.0 MHz relative to carbon. About 200 mg of finely crushed samples were placed in a dedicated cell, and accumulations were performed by 10,000 times at a room temperature of 25 to 26° C. and a pulse repetition time was 5 second. The analysis result is shown in FIG. 5.

FIG. 5 shows the ¹³C-CP/MASNMR spectrum of humic acids (HA) and fulvic acids (FA) extracted from each of Canadian moss peat and weathered coal. In FIG. 5, in order to know the relative content of carbon types classified according to the bonding pattern, the δ0 to 230 range of the spectrum is divided into seven regions which are classified according to the carbon types, i.e., an unsubstituted aliphatic C (alkane, fatty acid) (δ0 to 50), N-alkyl (amino acid, peptide, protein), +methoxy C (δ50 to 60), aliphatic group C—O (especially hydrocarbon) (δ60 to 110), aromatic group C (δ110 to 150), phenolic C (δ150 to 160), carboxyl C (δ160 to 190), and ketone C=0 (δ190 to 230).

When the percentage to the total area of areas surrounded by the spectrum curve, boundary of each above-mentioned region, and the base line is calculated to determine a relative carbon content, which shows that there are some differences in the molecular structure of humic acid (HA) and fulvic acid (FA) due to differences in raw materials.

The humic acid originating from Canadian moss peat (CP-HA) has a larger amount of aliphatic group C and a smaller amount of aromatic group C compared with the humic acid originating from weathered coal (WC-HA). The fulvic acid originating from Canadian moss peat (CP-FA) has a larger amount of aliphatic group C-0 and a smaller amount of phenolic C, carboxyl C, and ketone C=0 compared with the fulvic acid originating from weathered coal (WC-FA). Moreover, both the humic acid and fulvic acid originating from Canadian moss peat has a larger amount of alkyl and methoxy groups compared with the humic acid and fulvic acid originating from weathered coal. This presumably results from that the humification process of moss peat is different from the oxidation process of coal.

(4) Molecular Weight Analysis

Measurement of molecular weight was performed using high performance chromatography (HPLC, Waters 600, product of Millipore Co.). TOHSOH TSKgel G 2000 SW_{XL}+G3000SW_{XL} for gel permeation chromatography was used for a column, and 0.05 mol L⁻¹ phosphate-buffer (pH 7) and 0.05 mol^{L-1} of sodium chloride were used for an eluent. 10 μL of sample which is dissolved in the eluent (0.1 mg m^{L-1}) was injected, and the absorbance of fraction liquid was measured at the wavelengths of 280 nm and 400 nm using Waters 490E (Millipore Co.). The measurement results are shown in FIG. 6.

FIG. 6 is a table substituting for a drawing which summarizes the physical properties analysis results of each fulvic acid extracted from each raw material of original underground brine, extracted underground brine, weathered coal, Canadian moss peat, and andosol. As shown in FIG. 6. the molecular weight of each fulvic acid extracted from each raw material of original underground brine, extracted underground brine, weathered coal, Canadian moss peat, and andosol was 740, 810, 920, 780, and 680, respectively, in this order.

Based on the molecular weight, elementary composition, and attribution results of hydrogen and carbon of each fulvic acid whose raw material is different from each other, the average molecular formula was obtained while assuming that fulvic acid is one molecule. Due to the fact that fulvic acid is a mixture, and the measurement of the absolute molecular weight is difficult, the molecular weight of the same fulvic acid varies depending on measurement methods. Thus, the exact chemical structure thereof does not exist at present. Therefore, the assumed average molecular formula of FIG. 6 is useful for distinguishing fulvic acids whose raw materials are different from each other.

(5) Analysis of the Content of Carboxyl Group

The carboxyl group of fulvic acid was quantitated by a known calcium acetate method (Blom et al., 1957). As a result, the content of carboxyl group of moss peat fulvic acid was 3.98 mmol/g.

(6) Electron Spin Resonance (ESR) Spectrum Analysis

Fulvic acid powder was put in a quartz tube with a diameter of 0.5 mm, and the free radical concentration contained in the fulvic acid was determined using an electron spin resonance (ESR) apparatus (BRUKER, ESR300E). The determination was performed using an Mn2+ standard marker, and the measurement of spin concentration was performed using a DPPH (1,1-Diphenil-2picrylhydrazyl, molecular weight of 394, ESR 3.1×10²⁰ spins/g). As a result, the free radical content of moss peat fulvic acid was 7.27×10¹⁵ spins/g.

### EXAMPLE 3

### <Antiallergic Effect Verification Experiment of Fulvic Acid>

Bioassay was performed for the purpose of elucidating the existence of antiallergic effect of fulvic acid and the specific action mechanism thereof. First, the characteristics of the fulvic acid originating from Canadian moss peat used for this bioassay will be described.

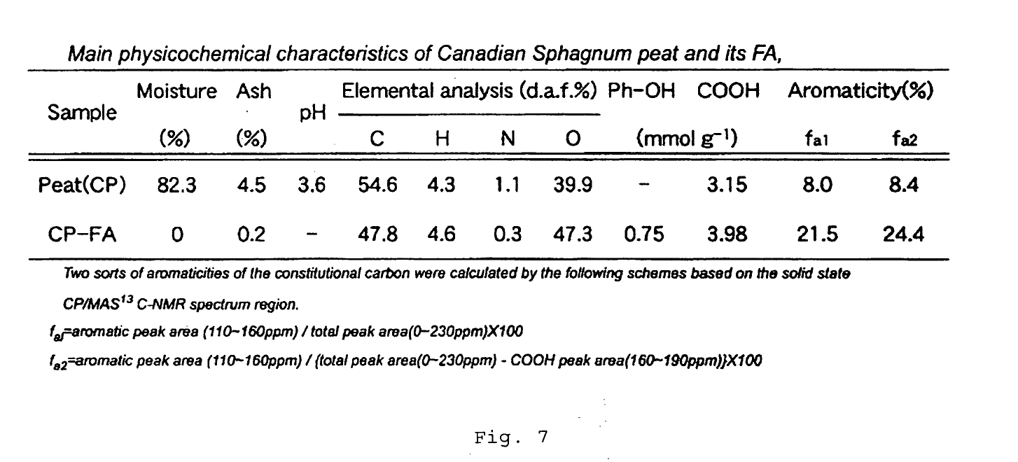

FIG. 7 is a table substituting for a drawing showing the characteristics of the fulvic acid originating from Canadian moss peat (CP-FA) used in the following bioassay.

The elementary composition (d.a.f %) of the fulvic acid originating from Canadian moss peat (CP-FA) is shown in FIG. 7. Carbon (C) was 47.8, hydrogen (H) was 4.6, nitrogen (N) was 0.3, oxygen (0) was 47.3, phenol hydroxyl group (Ph—OH) was 0.75 mmolg⁻¹, and carboxyl group (COOH) was 3.98 mmolg⁻¹.

The fₐ₁” and “fₐ₂” each shown in FIG. 7 represent an index showing the degree of aromatization of a humic substance. The “fₐ₁” is calculated based on a ratio of the aromatic group C content to a total carbon content from ¹³C-NMR spectrum, and the “fₐ₂” is calculated based on a ratio of the aromatic group C to the content of carbons rather than carboxyl C. When the fₐ₁ and fₐ₂ values are higher, humification is further progressed. As shown in FIG. 7, the fulvic acid originating from Canadian moss peat (CP-FA) is a component in which humification of the moss peat is progressed.

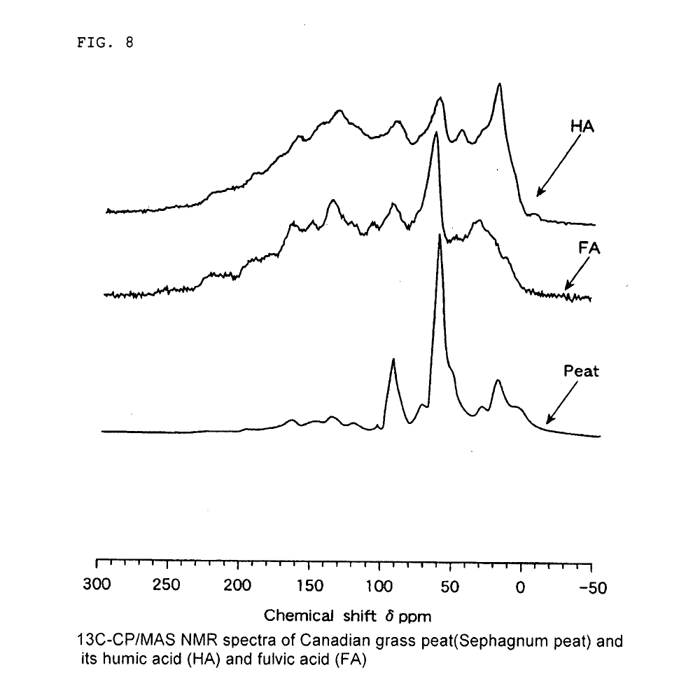

FIG. 8 is view (graph) showing ¹³C-CP/MASNMR spectrum of humic acid (HA) and fulvic acid (FA) of Canadian moss peat.

FIG. 8 shows that the humic acid (HA) and fulvic acid (FA) are decomposed into many fractions compared with the Canadian moss peat (Peat) as a raw material. This is presumably caused by disintegration of hydrogen chloride and sodium hydroxide in an extraction stage.

FIG. 8 also shows that the fulvic acid (FA) which is a water-soluble component of moss peat has a larger amount of alkyl and methoxy group compared with the humic acid (HA) which is an alkali soluble acid insoluble component, and the content of aromatic group and the content of carbohydrate are higher in the fulvic acid (FA) which is a water-soluble component of moss peat. Hereinafter, the procedure of the bioassay of Example 3 will be described.

(1) Preparation of a Fulvic Acid Sample

The fulvic acid extracted was melted in distilled water so as to yield a concentration of 1 mg mL⁻¹, and was sterilized with a 0.22 μm filter (Millipore, Japan).

(2) Cell and Cell Culture

Rat basophilic leukemia cell RBL-2H3 cells were used as a cell. The RBL-2H3 cells were cultured using a 10% Fetal bovine serum (FBS), 2 mML-glutamine, and 60 mg L⁻¹ kanamycin-containing Minimum Essential Medium Eagle (MEM, Nissui).

(3) β-hexosaminidase Release Measurement

It is known in the type I allergy reaction occurring in mast cells that the binding of an antigen and an antibody directly causes intracellular organelle flow, such as histamine, β-hexosaminidase, etc. For this reason, the influence of the fulvic acid (CP-FA) on the binding of an antigen and an antibody was confirmed by measuring a β-hexosaminidase release inhibition rate.

The above-mentioned RBL-2H3 cells cultured beforehand in MEM culture medium was prepared so as to be 5.0×10⁵ cells mL⁻¹, and 1 mg mL⁻¹ of mouse anti-DNP IgE antibody (Sigma, Co.) was added thereto to yield a final concentration of 0.3 μg mL⁻¹, followed by sufficiently suspending.

100 μL well⁻¹ of the resultant was seeded in a 96 well plate (Falcon Co.), and placed in an incubator having a temperature of 37° C. and a CO₂ concentration of 5% one night for cell sensitization. On the next day, the cells were washed twice with 0.02% EDTA·2Na/PBS(−)100 μL well⁻¹. Then, 60 μL well⁻¹ of Releasing Mixture (116.9 mM NaCl, 5.4 mM KCl, 0.8 mM MgSO₄·7H₂O, 5.6 mM Glucose, 25 mM HEPES, 2.0 mM CaCl₂, 1 mg mL⁻¹ BSA, pH 7.7) was added. Further, 5 μL well⁻¹ of each sample (with FA adjusted to be 0.01, 0.1, 1.0, 10.0 μg mL⁻¹) was exposed. The resultant was allowed to stand in a CO₂ incubator for 10 minutes. At this time, 6.3 mM ketotifen (Sigma, Co.) was added in place of the sample as a positive control, and sterilized water was added as a negative control. 10 minutes later, 4 μL mL⁻¹ of antigen and 5 μL well⁻¹ of DNP-BSA (Cosmobio) were added, and the resultant was placed in an incubator having a temperature of 37° C. and a CO₂ concentration of 5% for 1 hour. After ice-cooling for 10 minutes and stopping the reaction, 20 μL of supernatant liquid was moved to a new 96 well plate. To the resultant was added 80 μL well⁻¹ of a substrate solution (5 mM 4-Nitrophenyl N-acetyl-β-D-glucosaminide dissolved in 50 mM-citric acid buffer) for reaction for 30 minutes in an incubator having a temperature of 37° C. and a CO₂ concentration of 5%.

200 μL well⁻¹ of reaction stop solution (0.1M NaHO₃, pH 10.0) was added to the reaction solution for stopping the reaction. Then, the absorbance was measured with a microplate reader at 405 nm (Powerscan HT, Dainippon Pharmaceutical Co., Ltd.).

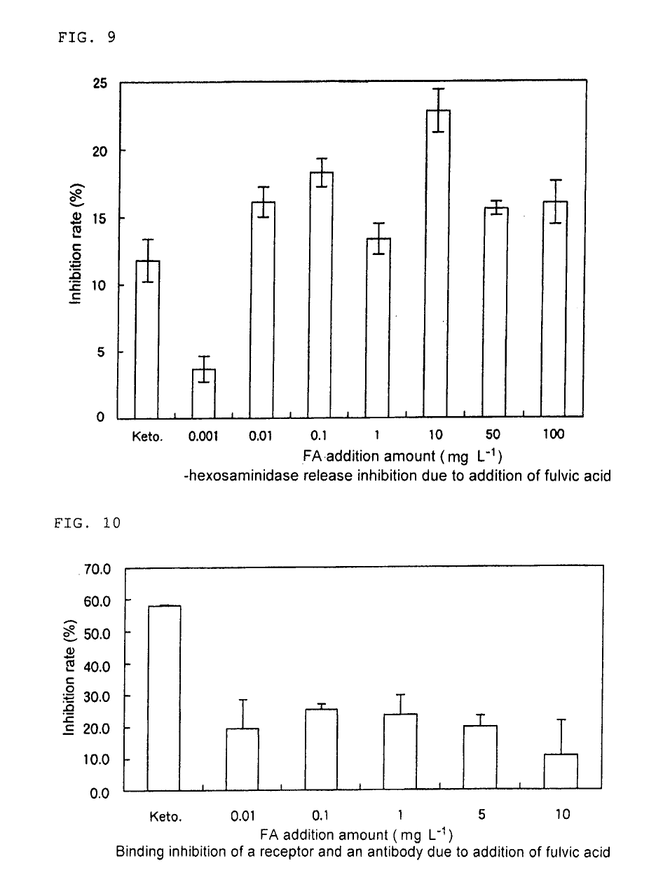

FIG. 9 is a view showing the measurement results, and is a view (graph) showing the results of investigating the influence of fulvic acid (CP-FA) on β-hexosaminidase release inhibition rate.

The axis of ordinates of FIG. 9 represents β-hexosaminidase release inhibition rate (%) and the axis of abscissa represents the concentration (μg mL⁻¹) of fulvic acid (CP-FA), respectively. The “Keto.” in FIG. 9 shows ketotifen (positive control) practically used as a clinical medicine for a comparison.

As shown in FIG. 9, fulvic acid (CP-FA) showed high β-hexosaminidase release inhibitory effect. In particular, when fulvic acid (CP-FA) was added in an amount of 10 μg mL⁻¹, the antiallergic effect was approximately twice that of ketotifen. Even when the addition concentration was higher or lower than the amount, the antiallergic effect fulvic acid (CP-FA) was equivalent to or higher than that of ketotifen.

Therefore, it was confirmed that the fulvic acid (CP-FA) has high concentration dependency in β-hexosaminidase release inhibition, and is useful as an antiallergic agent for inhibiting the antigen sensitization phase of the type I allergy reaction.

(4) Measurement of Binding Inhibition of a Receptor and an Antibody

Fulvic acid (CP-FA) was added in the antibody sensitization phase, and the effect of inhibiting release of the intracellular organelle due to binding inhibition of a receptor and an antibody on a cell membrane was investigated.

The RBL-2H3 cells cultured beforehand in MEM culture medium were prepared to be 1.0×10⁶ cells mL⁻¹, and 50 μL well⁻¹ of the resultant was seeded. Further, 5 μL well⁻¹ of each sample (with FA adjusted to be 0.01, 0.1, 1.0, 10.0 μg mL⁻¹) was exposed. The resultant was subjected to cell sensitization in an incubator with a CO₂concentration of 5% and a temperature of 37° C. for 1 hour. Thereafter, 1 mg mL⁻¹ of mouse anti-DNP IgE antibody (Sigma, Co.) was added thereto to yield a final concentration of 0.6 μg mL⁻¹, followed by sufficiently suspending.

On the next day, the cells were washed twice with 0.02% EDTA·2Na/PBS(−)100 μL well⁻¹. Then, 60 μL well⁻¹ of Releasing Mixture (116.9 mM NaCl, 5.4 mM KCl, 0.8 mM MgSO₄·7H₂O, 5.6 mM Glucose, 25 mM HEPES, 2.0 mM CaCl₂, 1 mg mL⁻¹ BSA, pH 7.7) was added. The resultant was allowed to stand in an incubator with a temperature of 37° C. and a CO₂ concentration of 5.0% for 10 minutes. 4 μL mL⁻¹ of antigen and 5 μL well⁻¹ of DNP-BSA were added, and the resultant was placed in an incubator having a temperature of 37° C. and a CO₂ concentration of 5.0% for 1 hour. After ice-cooling for 10 minutes and stopping the reaction, 20 μL of supernatant liquid was moved to a new 96 well plate. To the resultant, 80 μL well⁻¹ of a substrate solution (5 mM 4-Nitrophenyl N-acetyl-β-D-glucosaminide dissolved in 50 mM-citric acid buffer) was portioned for reaction at 37° C. for 30 minutes in an incubator with a CO₂ concentration of 5.0%.

200 μL well⁻¹ of reaction stop solution (0.1M NaHO₃, pH 10.0) was added to the reaction solution for stopping the reaction. Then, the absorbance was measured with a microplate reader at 405 nm (Powerscan HT, Dainippon Pharmaceutical Co., Ltd.).

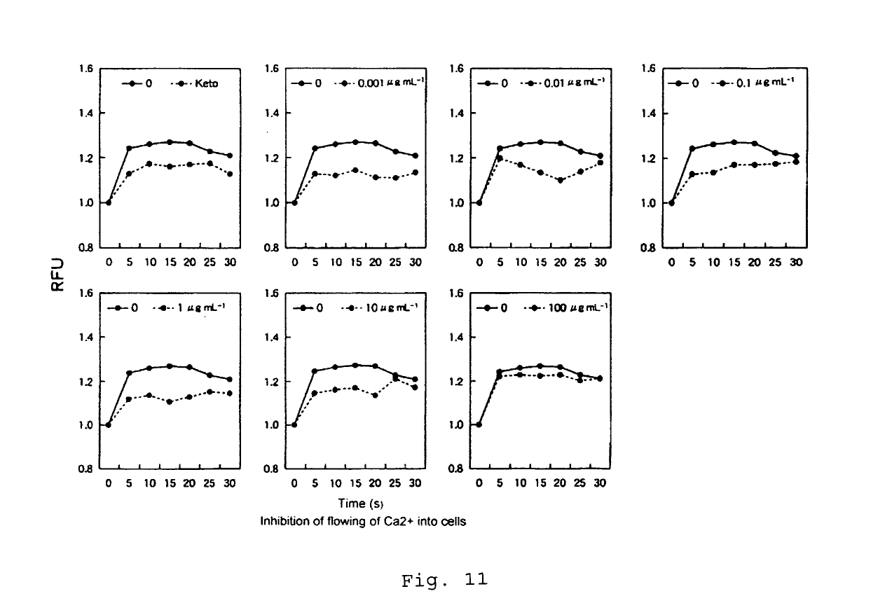

FIG. 10 is a view (graph) showing the results of measuring the absorbance. As shown in FIG. 10, release inhibition of β-hexosaminidase from mast cells was observed in fulvic acid (CP-FA). However, the release inhibition level of fulvic acid (CP-FA) was lower compared with ketotifen as a positive control, and the concentration dependency was not observed. This clarified that fulvic acid (CP-FA) is able to be expected to have a certain level of antiallergic effect also in the antibody sensitization phase of the type I allergy reaction.

(5) Measurement of the Concentration of Intracellular Calcium Ions (Ca²⁺)

Intracellular Ca²⁺ concentration was measured using a Calcium Kit-Fluo 3. The above-mentioned RBL-2H3 cells cultured beforehand in MEM culture medium was prepared so as to be 1.5×10 cells mL⁻¹, and 1 mg mL⁻¹ of mouse anti-DNP IgE antibody (Sigma, Co.) was added thereto to yield a final concentration of 0.3 μg mL⁻¹, followed by sufficiently suspending. The resultant was placed in an incubator having a temperature of 37° C. and a CO₂ concentration of 5% one night for cell sensitization.

On the next day, the cells were washed twice with PBS (−) 200 μL well⁻¹. Then, 100 μL of Loading buffer was added and the resultant was placed in an incubator having a temperature of 37° C. and a CO₂ concentration of 5% for 1 hour. The cells were washed twice with PBS (−) 200 μL well⁻¹, and the remaining Fluo3-AM was removed.

100 μL well⁻¹ of Recording medium was added. Further, 8.3 μL well⁻¹ of each sample (with FA adjusted to be 0.01, 0.1, 1.0, 10.0 μg mL⁻¹) was exposed. The resultant was allowed to stand in an incubator having a temperature of 37° C. and a CO₂ concentration of 5.0% for 1 hour. At this time, 6.3 mM ketotifen (Sigma, Co.) was used in place of the sample as a positive control, and sterilized water was used as a negative control. 1 hour later, while adding 4 μL mL⁻¹ of antigen and 67 μL well⁻¹ of DNP-BSA (Cosmobio), the fluorescence was measured with a microplate reader at 485 nm and 530 nm (Powerscan HT, Dainippon Pharmaceutical Co., Ltd.).

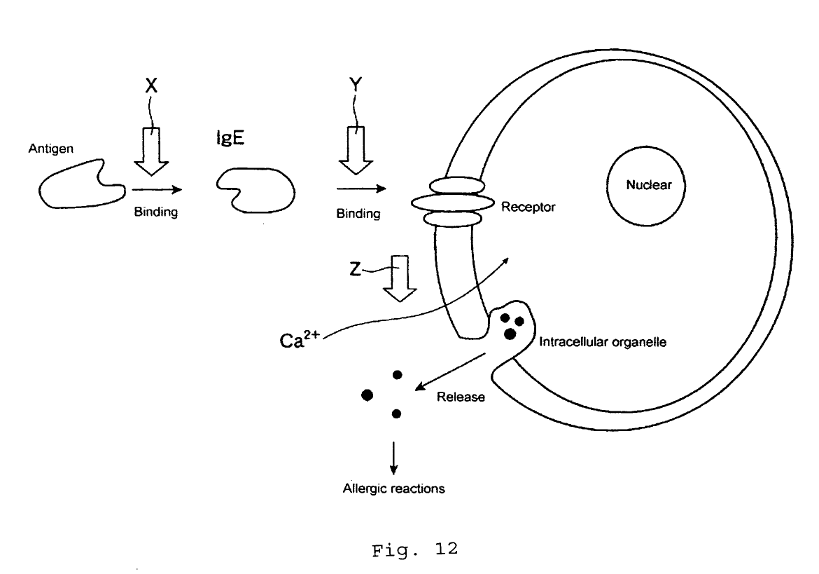

FIG. 11 is a view (graph group) showing the results of investigating the influence of fulvic acid on Ca²⁺ inflow inhibition due to antigen stimulus. The axis of ordinate of FIG. 11 represents the intracellular inflow ratio of Ca²⁺ due to antigen stimulus to intracellular Ca²⁺ with a concentration of 1.0 of control (addition of sterilized water). The axis of abscissa represents progress of the time (second) from the antigen stimulus.

It was clarified that the addition of each of the fulvic acid inhibited inflow of Ca²⁺ due to the antigen stimulus. Moreover, the activity was equivalent to ketotifen (positive control) as a Ca²⁺ inflow inhibitor, and it was confirmed that the Ca²⁺ inflow inhibitory effect worked immediately after the antigen stimulus. The concentration dependency was not observed in the Ca²⁺ inflow inhibitory effect of fulvic acid due to the antigen stimulus.

The results of Examples above showed that fulvic acid has the high antiallergic effect. FIG. 12 is a view for schematically illustrating the relationship between the developmental mechanism of the type I allergy and fulvic acid.

Fulvic acid acts on the developmental mechanism of the type I allergy in three phases: first, the antigen sensitization phase shown by arrow X in FIG. 12, secondly, the antibody sensitization phase shown by arrow Y in FIG. 12, and third, the degranulation phase (phase shown by arrow Z in FIG. 12) of cells caused by the inflow of calcium ions. Fulvic acid inhibits the onset of the type I allergy by inhibiting or intercepting these phases.

The present invention is useful for preventing and/or inhibiting the onset of the type I allergy. For example, the present invention can be widely applied to medical drugs and quasi medical drugs for the purpose of preventing and/or inhibiting the onset of the type I allergy, such as an orally administered agents, injection liquids, dermatological agents, poultices, etc.; cosmetics having a function of preventing and/or inhibiting the onset of the type I allergy, such as bath salts, skin care cosmetics (e.g., facial wash, facial cream, facial pack, etc.), etc.; foods and drinks, such as drinks (e.g., refreshing drink and the like), instant foods, confectioneries (e.g., candies, gums, etc.), breads, noodles, baby foods, seasonings, etc.; etc. Note that fulvic acid is approved as a food additive component by the Japan Ministry of Health, Labour and Welfare (Petition Docket No. 0601001).

1-13. (canceled)

14. A type I allergy inhibitor comprising, as an active ingredient, a therapeutically effective amount of fulvic acid preventing calcium ion from flowing into a cell due to antigen stimulus for inhibiting a degranulation phase.

15. The type I allergy inhibitor according to claim 14, wherein the fulvic acid is extracted from moss peat.

16. A composition comprising a therapeutically effective amount of at least fulvic acid inhibiting type I allergy by preventing calcium ion from flowing into a cell due to antigen stimulus for inhibiting a degranulation phase.

17. A facial wash, comprising the composition of claim 16.

18. A cosmetic, comprising the composition of claim 16.

19. Food and drink, comprising the composition of claim 16.

20. A dermatological agent, comprising the composition of claim 16.

21. A method for inhibiting onset of a type I allergy in a human by performing a specific hyposensitization treatment comprising administering to a human a therapeutically effective amount of fulvic acid for inhibiting an antigen sensitization phase and/or an antibody sensitization phase.

22. A method for inhibiting onset of a type I allergy in a human by performing a nonspecific hyposensitization treatment comprising administering to a human a therapeutically effective amount of fulvic acid for preventing degranulation.
